(19) United States
(12) Patent Application Publication
(10) Pub. No.: US 2014/0276880 A1
(43) Pub. Date: Sep. 18, 2014

(54) LOCKING ASSEMBLY FOR A SURGICAL DRILL BIT GUIDE

(71) Applicant: BLACKSTONE MEDICAL, INC., Lewisville, TX (US)

(72) Inventor: Nelson Li, Hillsdale, NJ (US)

(21) Appl. No.: 13/843,719

(22) Filed: Mar. 15, 2013

(51) Int. Cl. A61B 17/17

(52) U.S. Cl. CPC A61B 17/17
USPC 606/96

(57) ABSTRACT

The embodiments described and claimed herein relate to a locking assembly including a locking member adapted to set a maximum drilling depth for a drill bit, wherein the drill bit is adapted to extend through a drill bit guide. In one embodiment, the locking member includes a housing and a first inner sleeve, wherein the first inner sleeve has a resting unbiased position with respect to the drill bit guide. The locking member includes a first biasing member having a first end adapted to connect to a portion of the housing, and a second end adapted to connect to a portion of the first inner sleeve, wherein the first inner sleeve is resiliently rotatable to a temporary biased position upon rotation of the housing such that the first inner sleeve is adapted to rotate a circumferential distance with respect to the drill bit guide.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND

1. Technical Field

The embodiments described and claimed herein relate to a locking assembly for a drill bit guide adapted to be used for surgical drills and the like.

2. Background Art

Surgical drills of various types have long been employed in the medical arts. Typically, surgical drills are used to bore a hole into a bone, and are particularly useful in many different applications including bone and joint site implants, maxillofacial surgeries, cranial surgeries, and spinal surgeries. Many of these applications require precise control over drill bit penetration, and great care must be taken to ensure that the holes are drilled at precisely the correct place and precisely the correct depth. Accordingly, there is a need for a locking assembly that is adapted to be used in connection with a drill bit guide and/or a drill bit that would ensure that holes are drilled at the correct location and the correct depth.

### BRIEF SUMMARY

The embodiments described and claimed herein relate to a locking assembly that includes a locking member adapted to set a maximum drilling depth for a drill bit, wherein the drill bit is adapted to extend through a drill bit guide. In one embodiment, the locking member includes a housing and a first inner sleeve, wherein the first inner sleeve has a resting unbiased position with respect to the drill bit guide. The locking member includes a first biasing member having a first end adapted to connect to a portion of the housing, and a second end adapted to connect to a portion of the first inner sleeve, wherein the first inner sleeve is resiliently rotatable to a temporary biased position upon rotation of the housing such that the first inner sleeve is adapted to rotate a circumferential distance with respect to the drill bit guide. In one embodiment, the unbiased position comprises a locked position for the locking member with respect to the drill bit guide such that the housing is not adapted to move longitudinally with respect to the drill bit guide. In the same embodiment or in another embodiment, the temporary biased position comprises an unlocked position for the locking member with respect to the drill bit guide such that the housing is adapted to move longitudinally with respect to the drill bit guide.

In one embodiment, the drill bit guide is adapted to extend through the inner sleeve of the locking member, wherein the drill bit guide is adapted to remain stationary and not adapted to rotate with respect to the locking member. In one embodiment, the first biasing member further comprises at least one distal segment disposed between the first and second ends, wherein the at least one distal segment is adapted to loop around a portion of the first inner sleeve. In another embodiment, the biasing member includes a plurality of distal segments as adapted to loop around a portion of the first inner sleeve.

In one embodiment, the first biasing member comprises a spring. In another embodiment, the first biasing member comprises a coil. In yet another embodiment, the first biasing member comprises a spring coil.

In one embodiment, the first inner sleeve comprises an opening adapted to retain a first bearing feature, wherein the first bearing feature is adapted to engage with a corresponding locking surface on a drill bit guide. In one embodiment, the locking member further includes a first indent disposed on an inside surface of the housing, wherein the first indent is adapted to rotate along with the housing such that in the temporary biased position the first indent is aligned with the first bearing feature, wherein the alignment of the first indent with the first bearing feature allows the locking member to move longitudinally along the drill bit guide. In the same embodiment or in a different embodiment, the first inner sleeve comprises a shoulder adapted to engage with a retractable sheath, wherein a portion of the retractor is adapted to extend axially through the drill bit guide, wherein the shoulder is adapted to prevent the retractable sheath from the retracting axially into to the drill bit guide. In one embodiment, the retractable sheath has an extended position and a retracted position. In one embodiment, the retractable sheath's movement in and out of the drill bit guide is affected, at least in part, by a separate biasing member (a second biasing member) where in the second biasing member allows the retractable to move between the extended position and the retractable position.

Further, in one embodiment, the housing of the locking member includes a recessed cavity having a first circumferential length along an inside of the housing, wherein the first circumferential length is smaller or less than an inside circumference of the housing, wherein the recessed cavity is adapted to include an anchor adapted to connect to a portion of the first inner sleeve, wherein the anchor is adapted to move along the first circumferential length in the recessed cavity.

In one embodiment, the drill bit guide extends axially through the locking member. In some embodiment or in a different embodiment, the drill bit guide includes a plurality of locking surfaces, wherein the locking member is adapted to engage with at least one corresponding locking surface, wherein the locking member is adapted to longitudinally move along the drill bit guide from a first corresponding locking surface to a second corresponding locking surface, wherein the plurality of locking surfaces include a first locking surface and a second locking surface.

In another embodiment of a locking member that is adapted to set a maximum drilling depth for a drill bit, wherein the drill bit is adapted to extend through a drill bit guide, the locking member includes a housing having an inner surface and an outer surface, wherein the inner surface includes a recessed cavity, wherein the recessed cavity comprises a first circumferential length, a first inner sleeve disposed inside the housing and coaxially disposed with the housing, a first biasing member having a first end adapted to connect to a portion of the housing, a second end adapted to connect to a portion of the sleeve, and at least one distal segment adapted to loop around a portion of the first inner sleeve, and wherein the housing is resiliently rotatable in a circumferential direction such that the housing is adapted to rotate a circumferential distance to a temporary biased position, wherein the circumferential distance is less than or equal to the first circumferential length of the recessed cavity. In this embodiment, when the locking member is in the temporary biased position, the locking member is also in an unlocked position with respect to the drill bit guide such that the housing is adapted to move longitudinally along a portion of the drill bit guide. Further, in this embodiment, the housing of the locking member further includes a resting unbiased position, wherein in the resting unbiased position, the locking member is in a locked position with respect to the drill bit guide such that the housing is not adapted to move longitudinally along a portion of the drill bit guide. In this embodiment, the first biasing member comprises a spring coil that is coaxially disposed with respect to the inner sleeve such that a portion of the inner sleeve extends axially through the spring coil. In this embodiment, or in a different embodiment, the first biasing member comprises an unbiased position and a biased position, wherein the first biasing member is adapted resiliently return to the unbiased position. In this embodiment or in a different embodiment, the first inner sleeve comprises at least one indent, wherein the at least one indent is adapted to receive a corresponding bearing feature, wherein the bearing feature is adapted to engage with at least one locking surface on the drill bit guide. In this embodiment, or in another embodiment, the first inner sleeve includes a shoulder adapted to engage with a retractable sheath, wherein a portion of the retractor is adapted to extend axially through the drill bit guide, wherein the shoulder is adapted to prevent the retractable sheath from the retracting axially into to the drill bit guide. In one embodiment, the retractable sheath includes an extended position having a first protrusion length and a retracted position having a corresponding second protrusion length wherein the first protrusion length is greater than the second protrusion length.

In one embodiment, the drill bit guide include a plurality of locking surfaces, wherein the locking member is adapted to engage with at least one corresponding locking surface, wherein the locking member is adapted to longitudinally move along the drill bit guide from one corresponding locking surface to another corresponding locking surface.

In another embodiment, a locking member and drill bit guide combination is disclosed. In this embodiment, the drill bit guide includes a first stop collar, a second stop collar, a plurality of locking surfaces disposed between the first and second stop collars. In this embodiment, the locking member comprises a housing and a first inner sleeve, wherein the first inner sleeve is coaxially disposed with respect to the housing. In this embodiment, the first inner sleeve has a resting unbiased position with respect to the drill bit guide, a first biasing member having a first end adapted to connect to a portion of the housing, and a second end adapted to connect to a portion of the first inner sleeve, wherein the first inner sleeve is resiliently rotatable to a temporary biased position upon rotation of the housing such that the first inner sleeve is adapted to rotate a circumferential distance with respect to the drill bit guide. In this embodiment, the unbiased position comprises a locked position for the locking member with respect to the drill bit guide such that the housing is not adapted to move longitudinally with respect to the drill bit guide, wherein the temporary biased position comprises an unlocked position for the locking member with respect to the drill bit guide such that the housing is adapted to move longitudinally with respect to the drill bit guide.

In one embodiment, the locking member and drill bit guide combination further includes a retractable sheath, wherein the retractable sheath is adapted to extend in an axial direction through a portion of the drill bit guide, wherein the retractable sheath includes a corresponding stopping surface adapted to engage with a portion of the first inner sleeve.

Other embodiments, which include some combination of the features discussed above and below and other features which are known in the art, are contemplated as falling within the claims even if such embodiments are not specifically identified and discussed herein.

### BRIEF DESCRIPTION OF THE SEVERAL VIEW OF THE DRAWINGS

These and other features, aspects, objects, and advantages of the embodiments described and claimed herein will become better understood upon consideration of the following detailed description, appended claims, and accompanying drawings where:

FIG. 1 is a perspective view illustrating one embodiment of a locking assembly;

FIGS. 2A-2B depict perspective views of a locking assembly used in connection with a drill;

FIG. 3A depicts a side view of the locking assembly that is set to a first maximum drilling depth;

FIG. 3B depicts a side view of the locking assembly that is set to a second maximum drilling depth;

FIG. 4A is a perspective view of a locking member showing its inner components in a resting unbiased position;

FIG. 4B is a perspective view of one embodiment of the housing adapted to be used in connection with the locking member;

FIG. 4C is a longitudinal cross-sectional view of one embodiment of a locking assembly, showing the locking member from FIG. 4A in a locked position with respect to a drill bit guide;

FIG. 5A is a is a perspective view of one embodiment of the locking member and its inner components in a temporary biased position;

FIG. 5B is a longitudinal cross-sectional view of one embodiment of the assembly, showing the locking member from FIG. 5B in an unlocked position with respect to a drill bit guide;

FIG. 6 is a cross-sectional view of one embodiment of the locking member;

FIGS. 7A-7D are side-views of the locking assembly illustrating the operation of the sheath with respect to a drill bit disposed inside the sheath;

FIGS. 8A-8D illustrates the operation of the retractable sheath in connection with a locking member; and

FIG. 9 is an enlarged cross-sectional view of a portion of the drill bit guide.

It should be understood that the drawings are not necessarily to scale and that the embodiments are sometimes illustrated by graphic symbols, phantom lines, diagrammatic representations and fragmentary views. In certain instances, details which are not necessary for an understanding of the embodiments described and claimed herein or which render other details difficult to perceive may have been omitted. It should be understood, of course, that the inventions described herein are not necessarily limited to the particular embodiments illustrated. Indeed, it is expected that persons of ordinary skill in the art may devise a number of alternative configurations that are similar and equivalent to the embodiments shown and described herein without departing from the spirit and scope of the claims.

Like reference numerals will be used to refer to like or similar parts from Figure to Figure in the following detailed description of the drawings.

### DETAILED DESCRIPTION

Referring first to FIG. 1, a perspective view illustrating one embodiment of a locking assembly 10 is shown, wherein the assembly 10 is adapted to set a maximum drilling depth for a drill bit. In the embodiment shown, the assembly 10 includes a main body member 12 (also referred to as the main body 12 or a portion of a drill bit guide 12), a locking member 14, a retractable sheath 16, a proximal end 18 adapted to receive a drill bit, and a terminal end 20 through which the drill bit is adapted to protrude. Before describing the features of the assembly 10 more fully, the components typically associated with and used in connection with the assembly 10 should be noted. As shown in FIG. 2A, in one embodiment, the assembly 10 is adapted to be used in connection with a drill bit 42, which in turn is adapted to be connected to a drill 40.

Returning to FIG. 1, each of the components or features relating to assembly 10 in the embodiment are discussed in turn below.

Generally speaking, the main body member 12 is any type of tubular member that is adapted to guide a portion of a drill bit through it, and adapted to allow the locking member 14 to be adjusted along its length. In one embodiment, the body member 12 comprises a portion of drill bit guide, which is adapted to allow drill bit to extend therethrough. As shown in the embodiment of FIG. 1, the main body member 12 includes an entry portion 34, a first stop collar 22, a guide portion 26, an opening 27, a plurality of locking surfaces 24, a second stop collar 32, and an anchor 33. In the embodiment shown, the locking member 14 is adapted to lock in different fixed positions along the guide portion 26 between the first stop collar 22 and the second stop collar 32 by engaging with the plurality of locking surfaces 34. In one embodiment, guide portion 26 is a portion of body 12 that is disposed between the first and second stop collars.

In the embodiment shown, the entry portion 34 comprises a tubular segment that is adapted to receive a drill bit, and is adapted to allow the drill bit to extend through the main body 12. In the embodiment shown, the entry portion 34 and the first stop collar 22 are generally disposed near the proximal end 18, and the second stop collar 32 is generally disposed near the terminal end 20. In one embodiment, the first stop collar 22 and the second stop collar 32 are both fixedly attached to the main body 12 such that they do not rotate or move from their respective positions on the main body 12. In one embodiment, the first stop collar 22 is integrally formed as part of the body 12. In the same embodiment or in a different embodiment, the second stop collar 32 is attached to the body 12 by way of anchor 33 that fixedly attaches that second stop collar 32 to the body 12. In one embodiment, anchor 33 is a screw. In another embodiment, the anchor 33 is any type of fastener that is adapted to fixedly attach the second stop collar 32 to a portion of the guide portion 26 and/or body member 12.

As shown in FIG. 1, the main body 12 includes a guide portion 26. The guide portion 26 is generally disposed between the first stop collar 22 and the second stop collar 32, and is adapted to guide the locking member 14 across a plurality of fixed positions. As shown in FIG. 1, the guide portion 26 includes a plurality of corresponding locking surfaces 24 (also referred to as the locking surfaces 24). In one embodiment, the plurality of locking surface 24 includes one or more recessed grooves. Each recessed groove or corresponding locking surface 24 generally provides a surface for a bearing feature of locking member 14 to engage therein (e.g., bearing feature 66 shown in FIGS. 4-10). As such, the plurality of locking surfaces 24 allow the locking member 14 to be locked in different positions along the length of the body 12 between the first and second stop collars 22 and 32.

As shown in FIG. 1, the guide portion comprises at least one opening 27 along a top portion of guide portion 26 to allow the locking member 14 to be moved along the guide portion 26. In one embodiment or in another embodiment, the guide portion 26 includes a second opening (not shown in FIG. 1) that is generally disposed near the bottom portion of the guide portion 26. As shown in FIG. 1, the body member 12 is adapted to extend axially through the locking member 14.

Locking member 14 is explained more fully in connection with FIGS. 4-6, but it should be noted as a general matter that locking member 14 is any type of member that is adapted to lock the retractable sheath 16 at different depths or positions within the main body 12. In the embodiment shown, the locking member 14 is adapted to be locked at various fixed positions on the guide portion 26 between the first stop collar 22 and the second stop collar 32. In the embodiment shown, a portion of the guide portion 26 is coaxially disposed with respect to a portion of the locking member 14.

As shown in FIG. 1, in one embodiment, the locking member 14 includes a mounting member 28 and a knob 30 (also referred to as a rotary knob 30, twist-and-push-and-pull knob 30 or twist and push knob 30, or twist and pull knob 30).

It should be understood that locking member 14 is exemplary in nature, and may include other components or features not shown. For instance, in another embodiment, the locking member 14 does not include the mounting member 28. In such an embodiment, the locking member 14 simply comprises the knob 30.

As explained below in connection with FIGS. 4-6, the locking member 14, in one embodiment, has at least two positions relative to the body 12: a resting unbiased position, and a temporary biased adjustable position. In the resting unbiased position, the knob 30 is locked into at least one of the corresponding locking surfaces 24, and as such, is unable to move in a longitudinal direction along the guide portion 26. In the temporary biased adjustable position, the knob 30 is adapted to be in adjustable position, such that the knob 30 is adapted to move along the guide portion 26. In one embodiment, the temporary biased adjustable position is also referred to as the unlocked position given that the locking member 14 is able to move longitudinally along the guide portion 26 of the body 12, and the resting unbiased position is also referred to as the locked position given that the locking member 14 is unable to move longitudinally along the guide portion 26 and is adapted to be locked with respect to one of the plurality of corresponding locking surfaces 24. In one embodiment, the knob 30 is adapted to rotate with respect to the guide portion 26 to move from the resting unbiased position to the temporary biased adjustable position. In this embodiment, force is applied in one direction to the knob 30 which causes the knob 30 to move from the unbiased position to the biased position, and when the force is removed, the knob 30 rotates or springs back to the resting biased position. In this embodiment, rotational force is applied in one direction to the knob 30 which causes the knob 30 to move from the unbiased position to the biased position, and when the rotational force is removed, the knob 30 rotates or springs back to the resting biased position. In one embodiment, the knob 30 is twisted with respect to main body 12, thereby causing the locking member to move from the unbiased position to the temporary, biased position.

In one embodiment, the locking member 14 is resiliently rotatable. Resiliently rotatable means that the locking member 14 is adapted to rotate from the resting unbiased biased position to the temporary biased position when a first rotational force is applied to the locking member 14 in one direction, and then return to the resting unbiased position when the first rotational force is removed, and without the need to apply a second rotational force in the opposite direction. In one embodiment, the locking member 14 is adapted to include a first biasing member such that the first biasing member allows the locking member 14 to return to the resting unbiased position when the rotational force is applied to the knob 30.

Retractable sheath 16 is explained more fully in connection with FIGS. 7-8, but as a general matter, it should be understood that retractable sheath 16 is any tubular member disposed near the terminal end 20 of the assembly 10 that is adapted to engage with a portion of the locking member 14 such that it can be locked in several positions with respect to the main body 12. In one embodiment, the retractable sheath 16 is not adapted to rotate. Rather, it is adapted to retract and extend in an axial direction into or out of the body 12. In the embodiment shown, the sheath 16 includes an opening 36 through which a drill bit is adapted to protrude. The sheath 16, as shown, in FIG. 1, includes a window or opening 38 adapted to show the tip 50. Generally speaking, the window 38 allows a surgeon or operator of the drill 40 to view the tip 50 as it is being used to drill into a bone or body portion.

In one embodiment, the sheath 16 has an extended position that has a first protrusion that has a corresponding first protrusion length, and a retracted position that a second corresponding protrusion length. In one embodiment, the first protrusion length is greater than the second protrusion length.

In one embodiment, the sheath is adapted to connect to a second biasing member, wherein the second biasing member is adapted to bias the sheath in the extended position, such that axial force is to be applied for the sheath to retract to the retracted position. In one embodiment, when the axial force is removed the sheath moves back to the extended position. It should be understood. In one embodiment, the first biasing member operates independently from the second biasing member.

It should be understood that FIG. 1 is exemplary in nature, and may include other components or features, in addition to, or in place of the ones shown. For instance in another embodiment the locking member does not include a mounting member. In yet another embodiment, the guide portion 26 does not include the opening 26.

Turning next to FIG. 2A, a perspective view of the assembly 10 is shown in connection with a drill bit 42, which in turn is adapted to be connected to a drill 40. FIG. 2A illustrates an operational configuration in which the drill cover assembly 10 is used in connection with the drill bit 42, and FIG. 2B illustrates a disconnected configuration in which the assembly 10 is removed from the drill bit 42, and is not used in connection with the drill bit 42.

In the embodiment shown in FIGS. 2A-2B, the drill bit 42 includes a proximal segment 44, a distal segment, 45, a terminal segment 46, and a drill bit tip 50 (also referred to as the tip 50). Generally speaking, the proximal segment 44 is a segment on drill bit 42 that is disposed near the drill 40 (or in other words, disposed furthest away from the tip 50), and the terminal segment 46 is a segment or a portion of the drill bit 42 that is disposed near the tip 50 or closest to the tip 50 (or, in other words, disposed furthest away from the drill 40), and the distal segment 45 is generally a segment or portion of drill bit 42 that is generally disposed between the proximal segment 44 and the terminal segment 46. As shown in FIG. 2A, the proximal segment 44 and the distal segment 45 are generally covered by a drill bit guide 47 (also referred to as guide 47). In the embodiment shown, the drill bit guide 47 is connected to main body 12 of the assembly 10. In another embodiment, the drill bit guide 47 is integrally attached to the body 12 such that the body 12 comprises a portion of the drill bit guide 47 that is disposed near the terminal segment 46 of the drill bit 42. In yet another embodiment, drill bit guide 47 is adapted to attach to the body 12 via entry portion 34.

In the operational configuration shown in FIG. 2A, the assembly 10 provides a cover for the terminal segment 46 of the drill bit 42 such that only a portion of the drill bit 42 is adapted to protrude from the assembly 10 when drilling. As such, the assembly 10 controls the length of drill bit 42 that will be exposed for drilling, and therefore is adapted to provide a precise depth that the drill bit 42 will drill. To explain, as shown in FIG. 2B, the drill bit 42 has a fixed length 48, wherein the fixed length 48 of the drill bit 42 is the distance between the tip 50 and a point from where the drill bit 42 extends from the drill 40. Given that the length 48 is fixed, the locking member 14, in operation, is adapted to set a maximum distance that the retractable sheath 16 will retract into the body member 12, thereby allowing a portion of the terminal segment 46 of the drill bit 42 to protrude through the retractable sheath 16 when using the drill 40. The locking member 14 is adjustable in the longitudinal direction with respect to the body 12 or guide portion 26 such that the locking member is adapted to provide a plurality of maximum depths or distances that the sheath will retract into the body 12.

Turning next to FIGS. 3A-3B, a side view of the assembly 10 from FIG. 1 is shown, illustrating the locking member 14 being disposed at different locked positions between the first and second stop collars 22 and 32. As shown in FIG. 3A, the locking member 14 is disposed near the first stop collar 22, and as shown in FIG. 3B, the locking member 14 is deposed near the second stop collar 32. As such, the locking member 14 can be locked in a plurality of locked positions along the length of the main body 12 by engaging with one of the plurality of the locking surfaces 24. As shown in FIG. 3A, the guide portion 26 of body 12 includes a depth indicator portion 52. In one embodiment, the depth indicator 52 corresponds to the depth at which a drill bit 42 will be drilled into a bone. To explain by way of example, as shown in FIG. 3A, the locking member 14 is set a depth of “14 mm”. In this embodiment, the assembly 10 will ensure that the drill bit 42 is used to drill a hole that is 14 mm deep. In one embodiment, in this configuration, the sheath 16 is adapted to retract into the body 12 such that 14 mm of the drill bit 42 is adapted to protrude when drilling. In contrast to FIG. 3A, the locking member 14 in FIG. 3B has been moved all the way to the right and is set to a depth of “6 mm” As such, in this configuration, the assembly 10 will ensure that the drill bit 42 is used to drill a hole that is 6 mm deep. In one embodiment, in this configuration, sheath 16 is adapted to extract into the body 12 such that 6 mm of the drill bit is adapted to be exposed when drilling.

As shown in the embodiment of FIG. 3B, the plurality of corresponding locking surfaces 24 includes a first corresponding locking surface 27, a second corresponding locking surface 29, and a third corresponding locking surface 31. It should be understood that the plurality of locking surface 24 may include other additional locking surfaces not shown. As shown in FIG. 3B, the locking member 14 is adapted to engage with any of the first, second, or third corresponding locking surfaces 27, 29, and 31, such that each corresponding locking surface corresponds to a different maximum depth or length that a drill bit is adapted to protrude from the sheath 16 (e.g., when drilling).

Turning next to FIGS. 4A-4C and 5A-B, the locking member is now shown in greater detail. Before discussing these Figures in further detail, it should be noted that FIGS. 4A-4C show the locking member 14 in its unbiased position wherein the locking member 14 is in a locked position with respect to the body member 12 such that the locking member 14 is prevented from moving longitudinally along the body guide portion 26, while FIGS. 5A-5B show the locking member 14 in it temporary biased position, wherein the locking member 14 is adapted to move longitudinally along the length of the body guide 26.

With respect to FIGS. 4A-4C, FIG. 4A shows a perspective view of the knob 30 showing its inner components, FIG. 4B is a perspective view of the housing 54, and FIG. 4C is a cross-sectional view of the system 10, showing the knob 30 in connection with the main body member 12. As shown in FIG. 4A, the knob 30 includes a housing 54, a rotatable first inner sleeve 56, a second inner sleeve 58, a first biasing member 60, an anchor 62, a recessed cavity 63 in the housing 54, at least one indent 61 adapted to receive a bearing member 66, a hold 96 adapted to engage with the bearing member 66, a groove 68 adapted to secure a portion of the first biasing member 60, a first anchor 70 and a second anchor 72, a first end 74, and a second end 76.

As shown in FIG. 4A, the mounting member 28 is connected to the knob 30. In one embodiment, the mounting member 28 remains in a fixed position with respect to the guide portion 26, and does not rotate along with the knob 30. In another embodiment mounting member 28 rotates along with knob 30.

As shown in FIGS. 4A and 4C, the first inner sleeve 56 is coaxially disposed with respect to the second inner sleeve 58, which in turn is coaxially disposed with respect to the housing 54. In the embodiment shown, both the first inner sleeve 56 and the second inner sleeve 58 are disposed within the housing 54, such that the first inner sleeve 56 extends in an axial direction from the first end 74 towards the second end 76. In one embodiment, a portion of the second inner sleeve 58 also extends in an axial direction from the first end 74 towards the second end 76. The second inner sleeve 58 is adapted to connected to the housing 54 by way of the first anchor 70 and the second anchor 72. In one embodiment, as shown in FIG. 4C, the first inner sleeve 56 includes a first surface 86 facing the guide portion 26 and a second surface 90 facing the second inner sleeve 58. As shown, the first surface 86 is adapted to rest around a portion of the guide portion 26, and first a portion of the second surface 90 is adapted to connect with a portion of the first biasing member 60, and a second portion of the second surface 90 is adapted to connect with second inner sleeve 58.

In one embodiment, the second inner sleeve 58 comprises a ring. In another embodiment, second inner sleeve 58 comprises a tubular segment.

As shown in FIG. 4A, the rotatable first inner sleeve 56 comprises a tubular sleeve that is adapted to extend axially between the first end 74 and the second end 76 of the housing along a segment or portion of the guide portion 26, and is adapted to rotate with respect to the guide portion 26. In one embodiment, the inner sleeve 56 is adapted to be rotated around a portion of the guide portion 26 such that rotation is controlled by the first biasing member 60, and the second inner sleeve 58 comprises a ring-shaped member that is adapted to envelope or circumscribe the first inner sleeve 56.

As shown in FIG. 4A, a portion of the first inner sleeve 56 is adapted to connect to the anchor 62, which in turn is disposed within the recessed cavity 63. It should be understood that while anchor 62 is shown as a separate structural feature from the first inner sleeve 56 in the embodiment shown in FIG. 4A, it need not be a separate structural feature. As such, in another embodiment, the anchor 62 is integrally part of or integrally attached to the first inner sleeve 56. In yet another embodiment, the anchor 62 is a portion of the first inner sleeve 56 that extends into the recessed cavity 63. In one embodiment, the anchor 62 comprises a portion of the first inner sleeve 56 that is adapted to connect with one end of the first biasing member 60.

As shown in FIG. 4B, in one embodiment, the recessed cavity 63 includes a first circumferential length 67 along an inside surface of the housing 58 and a first stopping feature 65, and a second stopping feature 69. In the embodiment shown, the first circumferential length 67 is less than the circumference of the inner surface 73 of the housing 58. In one embodiment, the first stopping feature 65 is a first protrusion, the second stopping feature 69 is a second protrusion, wherein each of the first and second protrusions extends from the inner surface 73. The first stopping feature 65 and the second stopping feature are each adapted to prevent the anchor 62 from moving circumferentially beyond each of the first and stopping features 65 and 69.

As shown in FIG. 4B, the anchor 62 is adapted to be disposed within the recessed cavity 63 such that it is adapted to move along an inner circumference of the recessed cavity 63. As shown, the anchor 62 comprises a second circumferential length 71, wherein the second circumferential length 71 of the anchor 62 is less than the first circumferential length 67 of recessed cavity 63. As such, the anchor 62 is adapted to move in a circumferential direction between the first stopping feature 65 and the second stopping feature 69 along the first circumferential length 67. In the embodiment shown, the movement of the anchor 62 inside the cavity 63 is limited by the first and second stopping features 65 and 69.

In the embodiment shown, the first inner sleeve 56 is adapted to rotate a first maximum circumferential distance with respect to the guide portion 26. In one embodiment, the first maximum circumferential distance corresponds to the distance that anchor is able to move within the cavity 63. In this embodiment, the first maximum circumferential distance is the difference between first circumference length 67 and second circumference length 71.

In one embodiment, the first biasing member 60 includes a first end adapted to connect to the groove 68, a second end adapted to connect to the anchor 62, which in turn is adapted to connect to the first inner sleeve 56. In this way, the first inner sleeve 56 is adapted to rotate along with the housing 54, such that when the housing 54 rotates in a given direction, the first inner sleeve 56 also rotates in the same direction. In the same embodiment or in another embodiment, the first biasing member includes a first end adapted to connect to the groove 68, a second end adapted to connect to the anchor 62, and at least one distal segment that is adapted to loop around a portion of the first inner sleeve 56. As such, the first inner sleeve 56 is adapted to rotate along with the housing 54, such that when the housing 54 rotates in a given direction, the first inner sleeve 56 also rotates in the same direction. In yet another embodiment, the first biasing member 60 includes a plurality of distal segments, where at least one of the plurality at distal segments is adapted to loop around a position of the first sleeve 56. In one embodiment, the first biasing member 60 is a spring. In another embodiment the first biasing member 60 is a coil. In yet another embodiment the first biasing member 60 is a spring coil.

As shown in FIG. 4A, the first inner sleeve 56 includes an opening 96 adapted to engage with a first bearing feature 66. As shown in FIG. 4A, the inside of the housing includes at least one indent 61. When the first sleeve 56 is rotated to its biased position or state as shown in FIG. 5A, the indent 61 is aligned with the opening 96. In this position, the bearing member 66 is adapted to engage with the indent 64. The locking member 14 is then allowed to move in the longitudinal direction with respect to the guide portion 26.

In one embodiment, the inside surface 73 of the housing 54 includes a first indent 61 and a second indent (not shown). In one embodiment, the first and second indents are disposed diametrically opposite of each other. In one embodiment, first indent 61 and the second indent 64 are each recessed indentations disposed on the inside surface 73 such that each indentation is adapted to engage with a corresponding bearing feature. In one embodiment, the first indent 61 is adapted to engage with a first bearing feature 66, and a second indent is adapted to engage with a second bearing feature 77. Each bearing feature is adapted to engage in its respective opening such that the first bearing feature 66 is adapted to retained in hole or opening 96, and a second bearing feature 77 is adapted to be retaining in hole or opening 67. In one embodiment, the first and second bearing features 66 and 77 are each ball bearings.

As shown, FIG. 4C illustrates the locking member 14 in its unbiased position. In this position, the locking member 14 is locked into a corresponding locking surface 24 on the guide portion 26. Further, in this position, the indent 61 is not aligned with the opening 96 and the bearing member 66 and second indent (not shown) is not aligned with the opening 67 and the second bearing feature 77. As such, as shown in FIG. 4C, the locking member 14 is prevented from moving longitudinally, as the first bearing feature 66 is fully lodged or retained within the hole or opening 96 and the second bearing feature 77 is fully lodged or retained in hole 67.

In FIGS. 5A and 5B, the locking member 14 is in the temporary biased position. In this configuration as shown in FIG. 5A, the knob 30 is adapted to move longitudinally such that the indent 61 is aligned with the bearing feature 66, and the knob 30 along with mounting member 28 (i.e., locking member 14) is free to move in the longitudinal direction. As shown in FIG. 5B, the bearing feature 66 is allowed to enter the indent 61 in the housing 54, releasing the bearing member 66 from the locking surface 24 and allowing the locking member 14 to move longitudinally along the guide portion 26 of the body 12. In one embodiment, when the indent 61 is aligned with hole 96, the bearing feature moves into the indent 61.

As shown in FIGS. 4C and 5B, the body portion includes a second biasing member 78 that allows the retractable sheath 16 to move inside the body 12. In one embodiment, the first biasing members 60 operates independently of the second biasing member 78.

FIG. 6 is a cross-sectional view of the locking member 14 as viewed from the first end 74. As shown, the locking member 14 includes an outside diameter 80 and an inside diameter 82. The inside diameter 82 is generally approximately equal to the inside diameter of the of the inner sleeve 56. As shown in FIG. 6, the anchor 62 is disposed inside the cavity 63, and is adapted to circumferentially move between the first stopping feature 65 and the second stopping feature 69. In the embodiment shown in FIG. 6, the anchor 62 is rests along the first stopping feature 65. Although not shown in FIG. 6, a first end of a biasing member 60 is adapted to connect to the anchor 62 and a second end is adapted to connect to the groove 68, and at least one distal segment adapted to loop around at least once around the sleeve 56. It should be understood that the distal segment may be adapted to loop around more than once around sleeve 56. As shown in FIG. 6, the housing 54 is pinned or connected to second sleeve 56 by way of the first and second anchors 70 and 72. The second sleeve 58 is fixedly attached to the first sleeve 56 such that all three, housing 54, first sleeve 56 and second sleeve 58 are adapted to rotate in tandem with each other. In one embodiment, inner sleeve 58 is used to retain biasing member 60 within the housing 54.

FIGS. 7A and 7B illustrate a side-view of the assembly 10 showing the sheath 16 in its extended position and retracted position, respectively, when the locking member is disposed close to the proximal end of the main body 12. As shown in FIG. 7A, the sheath 16 is in the extended position having a first protrusion length 5. In this position, the sheath 16 is adapted to retract into the body 12 when force is applied such that the sheath 16 exposes a portion of the drill bit 42 that can drill a hole having a depth of 14 mm. In this regard, it should be noted that even though the locking member 14 is set at a particular depth, it does not mean that the tip 50 protrudes outside the sheath 16. Rather, the tip 50 is adapted to be retained inside the sheath 16. When axial force is applied against the tip 17 of sheath 16, the sheath 16 retract inwards to a second protrusion length 6 to expose segment 7 of drill bit 42, a depth position at which the locking member has been set. FIG. 7B shows the sheath 16 in its retracted position when axial force is applied to tip 17 of sheath 16. As shown, the sheath 16 retracts inwards to expose a segment 7 of the drill bit when axial force is applied to the sheath 16.

FIGS. 7C and 7D illustrate a side-view of the assembly 10 showing the sheath 16 in its extended position and retracted position when the locking member 14 is set to depth of 6 mm. Like FIG. 7A, the sheath 16 in FIG. 7C remains in its extended position having a first protrusion length 5. In FIG. 7D, the sheath 16 is adapted to be move to a retracted position when inward force is applied that will allow the sheath 16 to a retracted position, where in the sheath 16 retracts into the body 12 thereby exposing a segment 9 of the drill bit 42 when axial force is applied to the tip 17 of the sheath 16. As shown, the retractor has a protrusion length 8 in the retracted position.

As shown in FIGS. 7B and 7D, the exposed segment 9 in FIG. 7D is smaller than the exposed segment 7 in FIG. 7B.

FIG. 8A-8D illustrate the operation of the sheath 16 in connection with the locking member 14. It should be understood that sheath 16 moves independently of the locking member 14 but the locking member 14 prevents the sheath 16 from moving past a shoulder 100 disposed on inner sheath 56. As shown in FIG. 8A, first inner sleeve 56 includes a shoulder 100 and the sheath 16 includes a stop surface 102. As shown in FIG. 8A, the locking member 14 is set at the greatest depth position index such that the locking member 14 is pushed all the way back to the first stop collar 22. In this embodiment, the sheath 16 is shown to be fully extended. FIG. 8B illustrates the sheath 16 pushed back into the guide portion 26 when inward force is applied to the sheath 16. As shown in FIG. 8B, the shoulder 100 of the first inner sleeve 56 abuts the stop surface 102 of sheath 16 and prevents the sheath 16 from retracting any further, thereby defining the maximum retracted position of the sheath 16 when the locking member 14 is set at the greatest depth position index. FIG. 8C shows the locking member 14 set at the lowest depth position index such that the locking member 14 is positioned all the way forward to the second stop collar 32. In this embodiment, the sheath 16 is shown to be fully extended. FIG. 8D illustrates the sheath 16 pushed back into the guide portion 26 when inward force is applied to the sheath. The shoulder 100 of the first inner sleeve 56 again abuts the stop surface 102 of sheath 16 from retracting any further, thereby defining the maximum retracted position of the sheath 16 when the locking member 14 is set at the lowest depth position index.

FIG. 9 illustrates an enlarged cross-sectional view of a portion of assembly 10 with the locking member 14 removed, showing the first bearing feature 66 disposed within the opening 96. As shown, the opening 96 is an opening or hole in the first sleeve 56 having a depth 104 and width 106. In one embodiment, the hole 96 is cylindrical channel that extends from the first surface 86 of the first inner sleeve 56 to the second surface 90 of the first inner sleeve 56.

As shown in FIG. 9, the width 106 of the opening 96 is approximately equal to the width of the bearing feature 66 such that the bearing feature 66 does not fall through centrally downwards through the hole 96. Rather, as shown, the bearing 66 rests inside hole 96 such that it slightly protrudes through the opening 96 past the second surface 90. When the indent 61 is aligned with the opening 96 (shown in FIG. 5A), the bearing feature 66 is able to move into the indent 61, thereby allowing the locking member 14 to move longitudinally along the body portion 26 (i.e., an unlocked position). When the indent 61 is not aligned with the opening 96 (as shown in FIG. 4A), the bearing feature 66 is disposed or retained in the opening 66 such that the locking member 14 is prevented from moving longitudinally along the body portion 26 (i.e., an locked position).

Although the embodiments described and claimed herein have been described in considerable detail with reference to certain embodiments, one skilled in the art will appreciate that the inventions described and claimed herein can be practiced by other than those embodiments, which have been presented for purposes of illustration and not of limitation. Therefore, the spirit and scope of the appended claims should not be limited to the description of the embodiments contained herein.

1. A locking member adapted to set a maximum drilling depth for a drill bit, wherein the drill bit is adapted to extend through a drill bit guide, wherein the locking member comprises:
a housing and a first inner sleeve, wherein the first inner sleeve is coaxially disposed with respect to the housing, and wherein the first inner sleeve has a resting unbiased position with respect to the drill bit guide;
a first biasing member having a first end adapted to connect to a portion of the housing, and a second end adapted to connect to a portion of the first inner sleeve; and
wherein the first inner sleeve is resiliently rotatable to a temporary biased position upon rotation of the housing such that the first inner sleeve is adapted to rotate a circumferential distance with respect to the drill bit guide.

2. The locking member of claim 1, wherein:
the unbiased position comprises a locked position for the locking member with respect to the drill bit guide such that the housing is not adapted to move longitudinally with respect to the drill bit guide; and
the temporary biased position comprises an unlocked position for the locking member with respect to the drill bit guide such that the housing is adapted to move longitudinally with respect to the drill bit guide.

3. The locking member of claim 1, wherein the drill bit guide is adapted to extend through the inner sleeve of the locking member, wherein the drill bit guide is adapted to remain stationary and not adapted to rotate with respect to the locking member.

4. The locking member of claim 1, wherein the first biasing member further comprises at least one distal segment disposed between the first and second ends, wherein the at least one distal segment is adapted to loop around a portion of the first inner sleeve.

5. The locking member of claim 1, wherein the first biasing member comprises a spring coil.

6. The locking member of claim 1, wherein the first inner sleeve comprises an opening adapted to retain a first bearing feature, wherein the first bearing feature is adapted to engage with a corresponding locking surface on a drill bit guide.

7. The locking member of claim 1, further comprising:
a first indent disposed on an inside surface of the housing, wherein the first indent is adapted to rotate along with the housing such that in the temporary biased position the first indent is aligned with the first bearing feature, wherein the alignment of the first indent with the first bearing feature allows the locking member to move longitudinally along the drill bit guide.

8. The locking member of claim 1, wherein the first inner sleeve comprises a shoulder adapted to engage with a retractable sheath, wherein a portion of the retractor is adapted to extend axially through the drill bit guide, wherein the shoulder is adapted to prevent the retractable sheath from the retracting axially into to the drill bit guide.

9. The locking member of claim 1, wherein the housing includes a recessed cavity having a first circumferential length along an inside of the housing, wherein the first circumferential length is less than an inside circumference of the housing, wherein the recessed cavity is adapted to include an anchor adapted to connect to a portion of the first inner sleeve, wherein the anchor is adapted to move along the first circumferential length in the recessed cavity.

10. The locking member of claim 1, wherein the drill bit guide extends axially through the locking member.

11. The locking member of claim 1, wherein the drill bit guide include a plurality of locking surfaces, wherein the locking member is adapted to engage with at least one corresponding locking surface, wherein the locking member is adapted to longitudinally move along the drill bit guide from one corresponding locking surface to another corresponding locking surface.

12. A locking member adapted to set a maximum drilling depth for a drill bit, wherein the drill bit is adapted to extend through a drill bit guide, wherein the locking member comprises:
a housing having an inner surface and an outer surface, wherein the inner surface includes a recessed cavity, wherein the recessed cavity comprises a first circumferential length;
an first inner sleeve disposed inside the housing and coaxially disposed with the housing;
a first biasing member having a first end adapted to connect to a portion of the housing, a second end adapted to connect to a portion of the sleeve, and at least one distal segment adapted to loop around a portion of the first inner sleeve; and
wherein the housing is resiliently rotatable in a circumferential direction such that the housing is adapted to rotate a circumferential distance to a temporary biased position, wherein the circumferential distance is less than or equal to the first circumferential length of the recessed cavity.

13. The locking member of claim 12, wherein in the temporary biased position, the locking member is an unlocked position with respect to the drill bit guide such that the housing is adapted to move longitudinally along a portion of the drill-bit guide.

14. The locking member of claim 12, wherein the housing further comprises a resting unbiased position, wherein in the resting unbiased position, the locking member is in a locked position with respect to the drill bit guide such that the housing is not adapted to move longitudinally along a portion of the drill bit guide.

15. The locking member of claim 12, wherein the first biasing member comprises a spring coil that is coaxially disposed with respect to the inner sleeve such that a portion of the inner sleeve extends axially through the spring coil.

16. The locking member of claim 12, wherein the first biasing member comprises an unbiased position and a biased position, wherein the first biasing member is adapted resiliently return to the unbiased position.

17. The locking member of claim 12, wherein the first inner sleeve comprises at least one indent, wherein the at least one indent is adapted to receive a corresponding bearing feature, wherein the bearing feature is adapted to engage with at least one locking surface on the drill bit guide.

18. The locking member of claim 12, wherein the first inner sleeve comprises a shoulder adapted to engage with a retractable sheath, wherein a portion of the retractor is adapted to extend axially through the drill bit guide, wherein the shoulder is adapted to prevent the retractable sheath from the retracting axially into to the drill bit guide.

19. The locking member of claim 12, wherein the drill bit guide include a plurality of locking surfaces, wherein the locking member is adapted to engage with at least one corresponding locking surface, wherein the locking member is adapted to longitudinally move along the drill bit guide from one corresponding locking surface to another corresponding locking surface.

20. A locking member and drill bit guide combination comprising:
a drill bit guide comprising:
a first stop collar, a second stop collar, a plurality of locking surfaces disposed between the first and second stop collars;
a locking member comprising:
a housing and a first inner sleeve, wherein the first inner sleeve is coaxially disposed with respect to the housing, and wherein the first inner sleeve has a resting unbiased position with respect to the drill bit guide;
a first biasing member having a first end adapted to connect to a portion of the housing, and a second end adapted to connect to a portion of the first inner sleeve;
wherein the first inner sleeve is resiliently rotatable to a temporary biased position upon rotation of the housing such that the first inner sleeve is adapted to rotate a circumferential distance with respect to the drill bit guide;
wherein the unbiased position comprises a locked position for the locking member with respect to the drill bit guide such that the housing is not adapted to move longitudinally with respect to the drill bit guide; and
wherein the temporary biased position comprises an unlocked position for the locking member with respect to the drill bit guide such that the housing is adapted to move longitudinally with respect to the drill bit guide.

21. The locking member and drill bit guide combination of claim 20 further comprising:
a retractable sheath, wherein the retractable sheath is adapted to extend in an axial direction through a portion of the drill bit guide, wherein the retractable sheath includes a corresponding stopping surface adapted to engage with a portion of the first inner sleeve.
